(19) United States
(12) Patent Application Publication
(10) Pub. No.: US 2012/0109211 A1
(43) Pub. Date: May 3, 2012

(54) Vertebral column implant for stabilization and stiffening of vertebral bodies of a vertebral column

(76) Inventors: Reto Braunschweiler, Neftenbach (CH); Simon Fischli, Kriens (CH)

(21) Appl. No.: 13/137,890

(22) Filed: Sep. 20, 2011

(30) Foreign Application Priority Data
Oct. 1, 2010 (EP) 10186117.7

(51) Int. Cl. A61B 17/70

(52) U.S. Cl. 606/264

(57) ABSTRACT

A vertebral column implant for stabilization and stiffening of vertebral bodies of a vertebral column comprises first bone screws and second bone screws. First rigid connecting elements, are fixed in the first receiving means of the first bone screws. Second elastic connecting elements are fixed in second receiving means of the second bone screws. Each first connecting element is connectible to a second connecting element via coupling means. The coupling means are attached in a releasable way to a second bone screw, in which an end region of a second connecting element is held. The coupling means are connectible to an end region of a first connecting element. By means of this design, a rigid region of the vertebral column implant can be decoupled in a simple way from an elastic stabilizing region of the vertebral column implant.

This invention relates to a vertebral column implant for stabilization and stiffening of vertebral bodies of a vertebral column, comprising first bone screws, consisting of a screw-in portion which is screwable into a vertebral body, and first receiving means; second bone screws, consisting of a screw-in portion which is screwable into a vertebral body, and second receiving means; first connecting elements, which are rigid and are insertable in the first receiving means of the first bone screws and are able to be fixed therein; second connecting elements, which are elastic and are insertable in the second receiving means of the second bone screws and are able to be fixed therein; and coupling means, by means of which a first connecting element and a second connecting element in each case are connectible to one another.

Such vertebral column implants are known in diverse designs. Achieved with these vertebral column implants is that in the vertebral column a rigid stabilization of the vertebral bodies is made possible regionally, while in other regions the vertebral bodies are supported and stabilized by the elastic design of the system without a stiffening taking place. Desirable in the region of the rigid stabilization of the vertebral bodies is that an osseous growing together of the affected and stabilized vertebral bodies is achieved; with the elastic stabilization no stiffening of the vertebral bodies should occur.

Such a vertebral column implant for stabilization and stiffening of vertebral bodies of a vertebral column is known, for example, from EP-A 1961392.

If, after a certain time, the growing together and the ossification of the vertebral bodies with respect to one another, which have been reinforced or stiffened by the vertebral column implant, has progressed so far that inherent stability exists, the stiffening regions of the vertebral column implant are no longer needed; they are therefore rather superfluous and can interfere. It would thus make sense, after the respective growing together process, if these stiffening regions of the vertebral column implant could be completely decoupled from the elastic stabilizing region or if necessary even removed, while the regions of the vertebral column implant that serve the stabilization of the vertebral bodies should be kept in order to be able to further exercise this supporting and stabilizing effect. This requires that the stiffening region and the stabilizing and supporting region of the vertebral column implant can be decoupled from one another in a simple way, which is not made possible with the known vertebral column implants.

The object of the present invention thus consists in designing a vertebral column implant for stabilization and stiffening of vertebral bodies of a vertebral column in such a way that the stabilizing region, which has an elastic connecting element, and the rigid region with a rigid connecting element are connected together in such a way that they are decouplable from one another in the simplest way.

This object is achieved according to the invention in that the coupling means, with a first region, are able to be attached to a second bone screw in a releasable way, in which second bone screw an end region of a second connecting element is held, and the coupling means with a second region opposite the first region are connectible to an end region of a first connecting element.

By means of this solution the rigid region of the vertebral column implant is able to be decoupled from the stabilizing and elastic region in a simple way. The connection of the second bone screw to the end region of the elastic second connecting element remains during the surgical intervention. Thus nothing changes with respect to the stabilizing capability of this elastic region of the vertebral column implant.

Preferably the first region of the coupling means is provided with a tensioning element, which is insertable in a guided way into the second receiving means and via tensioning means, which are able to be put on the second receiving means, is able to be tensioned against the second connecting element inserted in the second receiving means. The coupling means is thereby able to be connected in a simple way to the second bone screw.

Preferably the second receiving means are designed U-shaped, and are composed of two arms, between which the bearing surface for the second connecting element is disposed and on which two arms the tensioning means, consisting of a locking element provided with a first tensioning screw, is able to be placed and locked therewith, which simplifies the procedure during the surgical intervention.

Preferably the tensioning element is provided with guide ribs, which, together with guide surfaces provided on the arms, form the guides, which results in a simple construction.

Another advantageous embodiment of the invention consists in that the second region of the coupling means is designed as a further tensioning means, in which an end region of a first connecting element is able to be fixed. During the respective surgical intervention, this additional separation possibility facilitates both the insertion and the decoupling of this region of the vertebral column implant.

An especially simple construction for this further tensioning means is achieved in that this further tensioning means has a U-shaped receiving part for the first connecting element and a threaded part, opposite the U-shaped receiving part, for receiving a second tensioning screw.

An improvement of the clamping of the first connecting element with the further tensioning means can be achieved in that a clamping element is inserted between the second tensioning screw and the first connecting element.

A further advantageous embodiment of the invention consists in that the tensioning element and the further tensioning means, which form the coupling means, are connected to one another in a releasable way, which facilitates manipulation during the respective surgical intervention.

Preferably the tensioning element has at least one nose-type projection, which projects into the further tensioning means, whereby a very simple construction for this connection is achieved.

Another advantageous embodiment of the invention consists in that the nose-type projection is provided with a threaded bore, into which a further tensioning screw is screwable, which nose-type projection projects between the second tensioning screw and the first connecting element of the further tensioning means. The position of the nose-type projection with respect to the connecting element can thereby be adjusted, whereby first connecting elements having different diameters can be used, for example.

A further advantageous embodiment of the invention consists in that the second connecting elements have a surface structure consisting of ridges and grooves running substantially transversely to the longitudinal axis, and in that the second receiving means and the surface of the tensioning means turned toward the second connecting element are provided with grooves and ridges that correspond to the ridges and grooves of the second connecting element. A formfitting connection can thereby be achieved in an optimal way between the second connecting element and the second receiving means of the second bone screw.

Embodiments of the present invention will be explained more closely in the following, by way of example, with reference to the attached drawings.

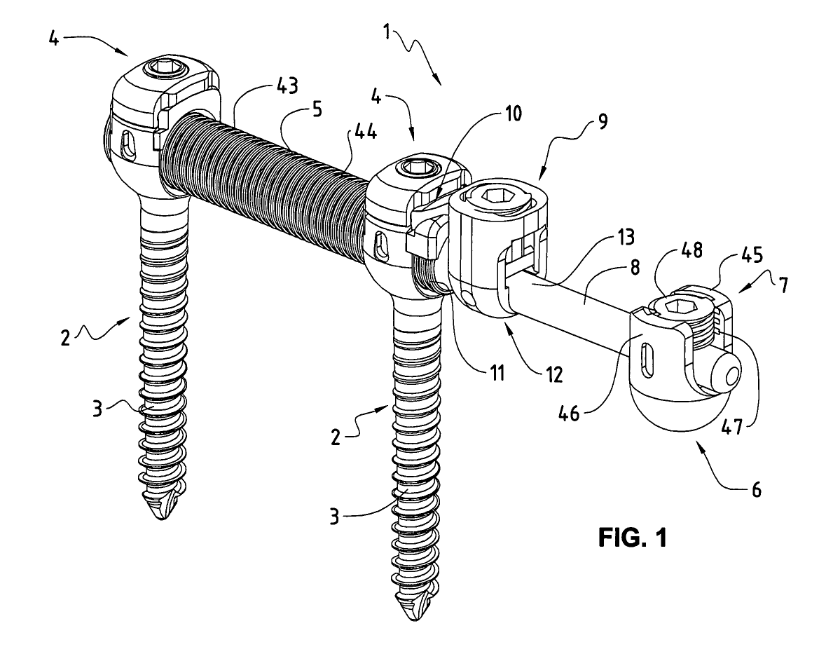

FIG. 1 shows in a three-dimensional representation a first embodiment of a portion of a vertebral column implant according to the invention;

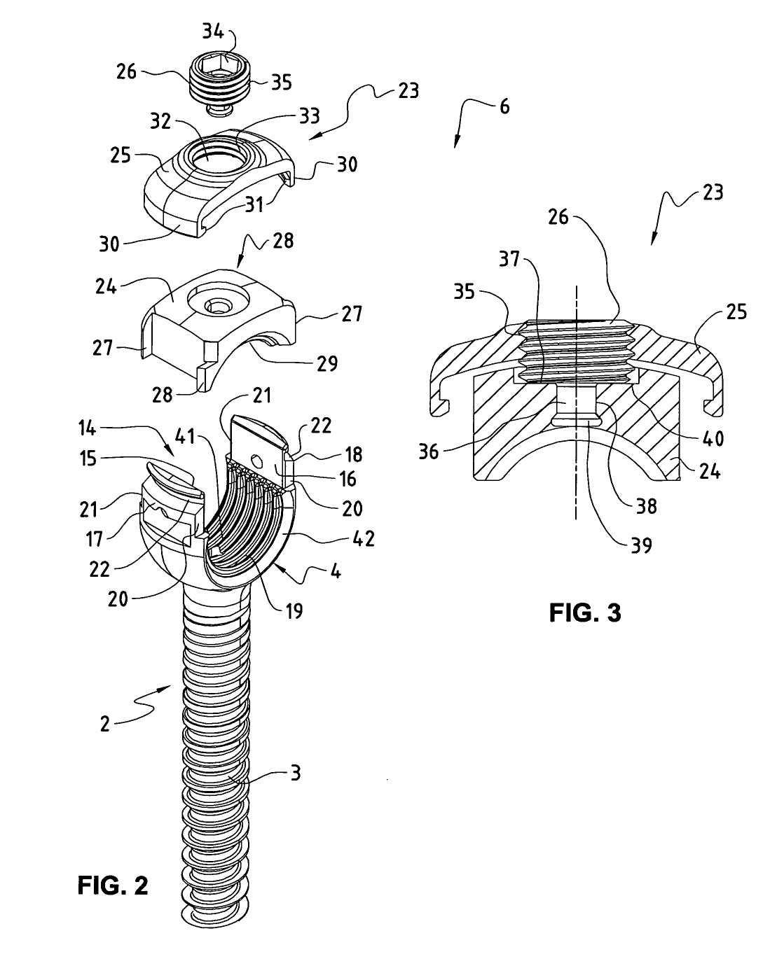

FIG. 2 shows in a three-dimensional representation a second bone screw with tensioning means of the vertebral column implant according to FIG. 1;

FIG. 3 shows a sectional view through the tensioning means according to FIG. 2;

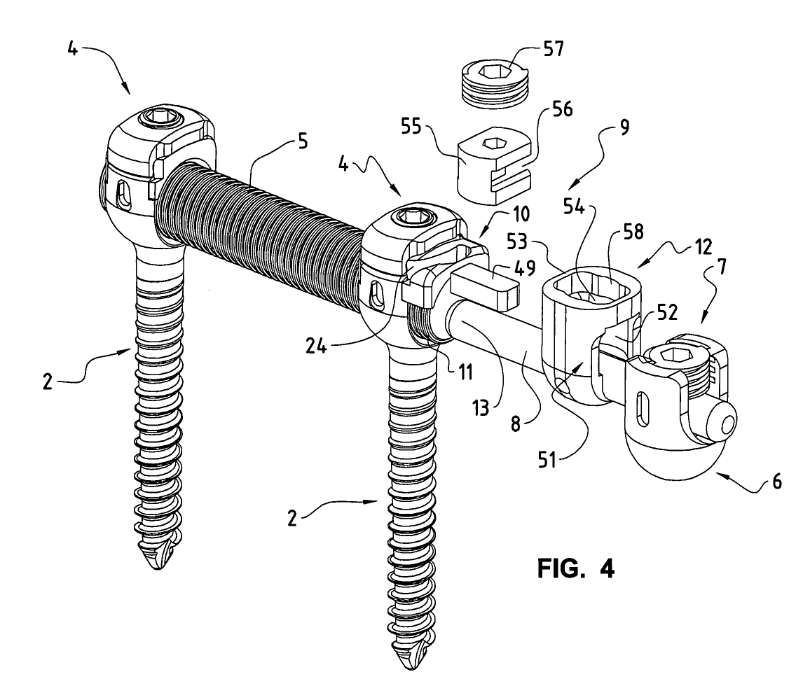

FIG. 4 shows in a three-dimensional representation the vertebral column implant according to FIG. 1 with coupling means illustrated with individual components;

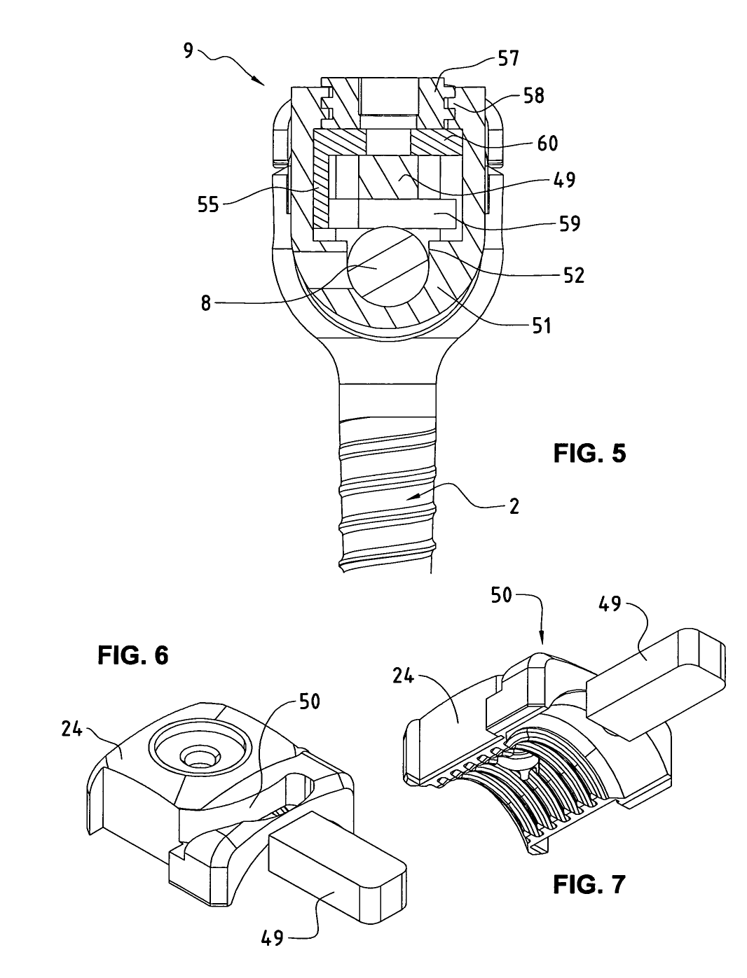

FIG. 5 shows a sectional view through the further tensioning means of the vertebral column implant according to FIG. 1;

FIG. 6 shows a view from above of the three-dimensionally represented tensioning element of the vertebral column implant according to FIG. 1;

FIG. 7 shows a view from below of the three-dimensionally represented tensioning element according to FIG. 6;

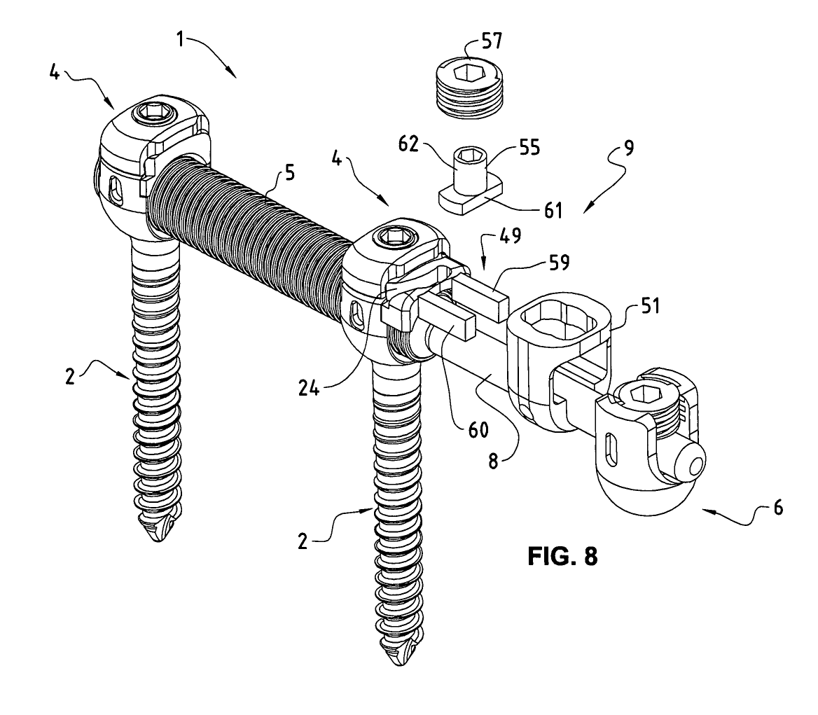

FIG. 8 shows in a three-dimensional representation a second embodiment of a portion of a vertebral column implant according to the invention with coupling means shown with individual components;

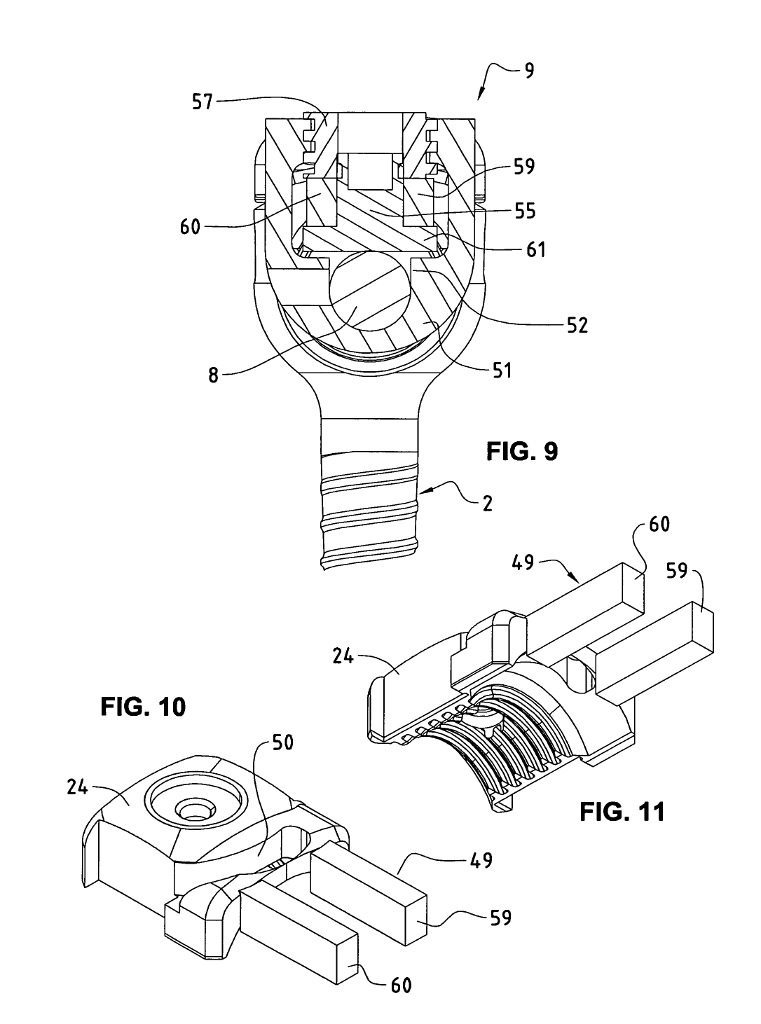

FIG. 9 shows a sectional view through the tensioning means of the vertebral column implant according to FIG. 8;

FIG. 10 shows a view from above of the three-dimensionally represented tensioning element of the vertebral column implant according to FIG. 8;

FIG. 11 shows a view from below of the three-dimensionally represented tensioning element according to FIG. 10;

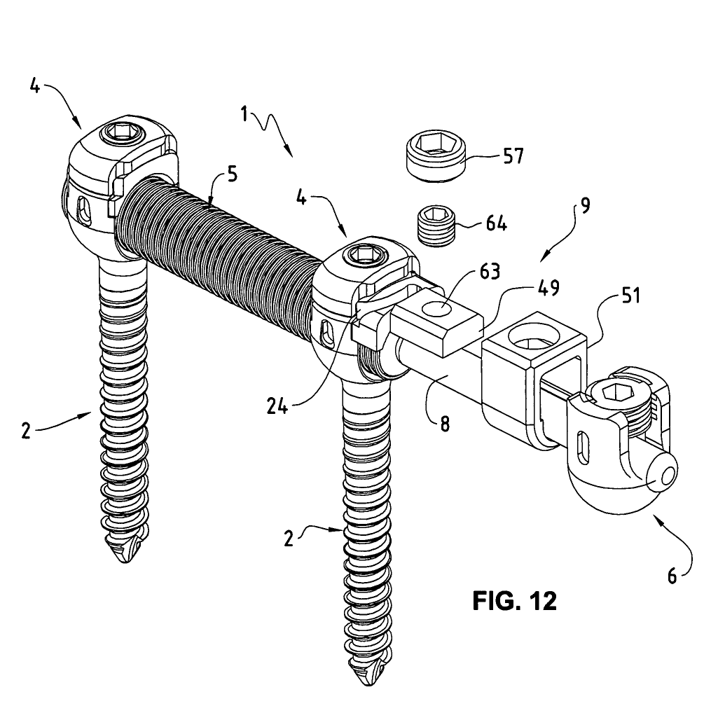

FIG. 12 shows in a three-dimensional representation a third embodiment of a portion of a vertebral column implant according to the invention with coupling means shown with individual components;

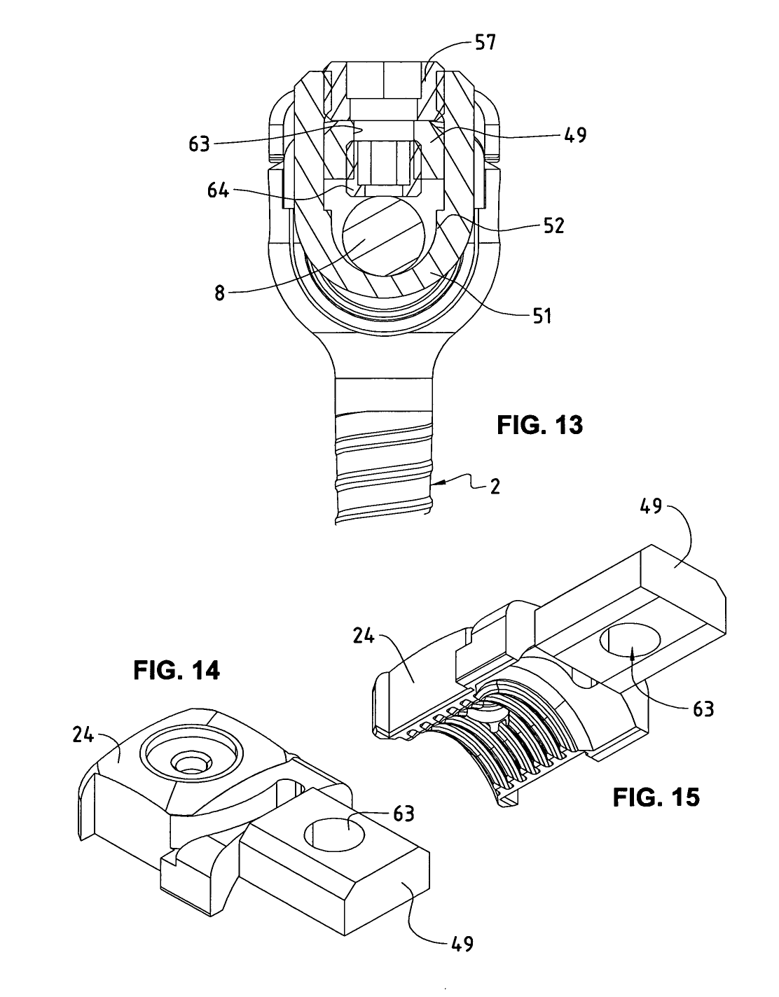

FIG. 13 shows a sectional view through the tensioning means of the vertebral column implant according to FIG. 12;

FIG. 14 shows a view from above of the three-dimensionally represented tensioning element of the vertebral column implant according to FIG. 12; and

FIG. 15 shows a view from below of the three-dimensionally represented tensioning element according to FIG. 14.

As can be seen from FIG. 1, such a portion of a vertebral column implant 1 according to the invention for stabilization and stiffening of vertebral bodies of a vertebral column is composed of second bone screws 2, which each comprise a screw-in portion 3 provided with a threading and second receiving means 4. In a known way, a second bone screw 2 of this kind with the screw-in portion 3 is screwable into a vertebral body of a vertebral column. Inserted and firmly held in the second receiving means 4 is a second connecting element 5. This second connecting element 5 is rod-shaped, and is made of an elastic material, for example a biocompatible polyurethane-based synthetic material.

The vertebral column implant 1 according to the invention further comprises first bone screws 6, which likewise have in a known way a screw-in portion, which is not shown in the figures, but which corresponds to the screw-in portion 3 of the second bone screw 2 and is correspondingly screwable into the intended vertebral body of the vertebral column. These first bone screws 6 are provided with first receiving means 7, in which a first connecting element 8, which is designed rod-shaped, can be inserted and fixed. This first connecting element 8 is rigid, and is made of a metallic material, for instance a titanium alloy.

The respective first connecting element 8 is connectible in each case to a second connecting element 5, adjacent thereto, via coupling means 9. These coupling means 9 are held in a releasable way on a second bone screw 2 by means of a first region 10, in which bone screw an end region 11 of the second connecting element 5 is likewise held. The second region 12, opposite the first region 10, of the coupling means 9 is connectible to an end region 13 of a first connecting element 8, which will be described later in detail.

Such a vertebral column implant 1 is adaptable in practically any desired way to the vertebral column to be stabilized. This vertebral column implant can be made up of rigid first connecting elements 8 and elastic second connecting elements 5, which co-operate with the respective first or second bone screws 6 or respectively 2. The respective connecting elements 8 and 5 can be cut to the desired length, and, by means of the corresponding combinations, the vertebral column can be stabilized in the desired regions in an elastic way and in other desired regions can be reinforced or stiffened, the corresponding bone screw being screwed into the intended vertebral body in each case.

The structure of the second bone screws 2 is shown in FIGS. 2 and 3. The second receiving means 4 connected to the screw-in portion 3 are provided with a U-shaped recess 14, which is formed by the inner surfaces 15, 16 of two arms 17, 18. The two inner surfaces 15 and 16 of these two arms 17 and 18 are connected together by a bearing surface 19, the inner surfaces 15 and 16 and the bearing surface 19 being adapted to the shape of the second connecting element 5 to be inserted in this U-shaped recess 14, in which recess ridges and grooves 41, 42 are provided, in which corresponding grooves 43 and ridges 44, provided on the second connecting element 5 (FIG. 1), engage in the inserted state, and thus a formfitting connection is formed.

The two lateral regions of the respective arms 17 and 18 are each provided with a guide surface 20, 21. Provided in the upper region on the outside on the two arms 17 and 18 is in each case a groove 22 running transversely to these arms. The bottom of the groove and the surfaces limiting the groove 22 are designed circular.

The U-shaped recess 14 of the second receiving means 4 of the second bone screw 2 is closed off by tensioning means 23. These tensioning means 23 are composed of a tensioning element 24, a locking element 25 and a first tensioning screw 26. The tensioning element 24 is able to be inserted between the two arms 17 and 18. For this purpose this tensioning element 24 is provided with two pairs of guide ribs 27 and 28, opposite one another, which together with the guide surfaces 20 and 21 of the two arms 17 and 18 form the guide. The surface 29 of the tensioning element 24 turned toward the bearing surface 19 likewise has a shape adapted to the shape of the second connecting element 5 (FIG. 1).

The locking element 25 has the shape of a bow, and is provided on the two end regions opposite one another with a collar 30 in each case. Provided on each of these collars 30 is a protrusion 31, which protrusions are directed toward each other. In addition, the locking element 25 is provided with a bore 32, which is provided with a threading 33. The first tensioning screw 26 can be screwed into the threading 33 of this bore 32, which tension screw is provided with a hexagonal recess 34. With these tensioning means 23 the U-shaped recess 14 is able to be closed in the second receiving means 4 of the second bone screw 2; the second connecting element placed in this U-shaped recess is thereby locked in place.

As can be seen from FIG. 3, the tensioning means 23 can be assembled into a unit. The first tensioning screw 26 is provided with a threaded part 35, to which a bolt piece 36 is added. Formed between the threaded part 35 and the bolt piece 36 is a support surface 37. The bolt piece 36 projects into a central bore 38, which is made in the tensioning element 24. The end region of the bolt piece 36 remote from the threaded part 35 is provided with a thickening 39, which is achievable, for example, through crimping of the end region of this bolt piece 36, which crimping procedure can be carried out after insertion of the first tensioning screw 26 in the tensioning element 24. The first tensioning screw 26 and the tensioning element 24 are thus connected to each other in a way rotatable about the bolt piece 36. The first tensioning screw 26 with the tensioning element 24 connected thereto can then be screwed into the locking element 25, whereby the tensioning means 23 is formed as a unit. For placement of the tensioning means 23 on the second bone screw 2, the locking element 25 is aligned transversely with respect to the tensioning element 24. The tensioning means 23 can then be moved into the U-shaped recess 14 of the second bone screw 2. The guide ribs 27 and 28 of the tensioning element 24 are guided by the guide surfaces 20 and 21 of the two arms 17 and 18. The locking element 25 can then be rotated about the axis of the first tensioning screw 26 over the respective guide rib 28, until the locking element 25 is located in the locked position, as can be seen from FIG. 1. The first tensioning screw 26 can then be tightened, and the second connecting element 5 is locked in place in the second bone screw 2.

As can be seen from FIG. 1, the first receiving means 7 of the first bone screw 6 consist in each case of two arms 45 and 46, which form a U-shape, in which the first connecting element 8 is placeable. Provided on the inner side of the arms 45 and 46 is a threading 47. Screwable into this threading 47 is also a tensioning screw 48, with which the first connecting element 8 is able to be locked in place in the first bone screw 6. Of course it would also be conceivable to design the first receiving means 7 of the first bone screw 6 differently in a known way, likewise in such a way that the first connecting element 8 is able to be locked in place in the first receiving means 7.

The structure of the coupling means 9 used in this first embodiment example is shown in FIGS. 4 to 7. FIG. 4 shows once again the vertebral column implant 1 according to FIG. 1 with first bone screws 6 and first receiving means 7, in which the first connecting element 8 is held, as well as second bone screws 2 with second receiving means 4, in which the second connecting element 5 is held.

The first region 10 of the coupling means 9 is made up of a tensioning element 24, as has been shown in FIGS. 2 and 3. This tensioning element 24 is part of the tensioning means 23, with which a second bone screw 2 is closable. The tensioning element 24 is provided here with a nose-type projection 49, which extends in longitudinal direction beyond the end region 11 of the second connecting element 5, and runs parallel to the end region 13 of the first connecting element 8. This tensioning element 24 with the nose-type projection 49 can be seen in detail in FIGS. 6 and 7. In order to make the locking element 25, which is shown in FIG. 2, rotatable with respect to this tensioning element 24, the tensioning element 24 is provided with a slot-shaped recess 50, which runs in a circular way, in the region of the nose-type projection 49.

As can be seen furthermore from FIG. 4, the second region 12 of the coupling means 9 is designed as further tensioning means 51, which has a U-shaped receiving part 52 for the first connecting element 8. The U-shaped receiving part 52 is closed off by a cap 53 which is provided with a through hole 54. Insertable into the further tensioning means 51 through this through hole 54 is a clamping element 55, which is rotatable in the further tensioning means 51, and is provided with a slot 56. The further tensioning means 51 is pushed onto the first connecting element 8, and the clamping element 55 is inserted into the further tensioning means 51 through the through hole 54 and is rotated, so that, when pushing this further tensioning means 51 with the inserted clamping element 55 toward the nose-type projection 49 of the tensioning element 24, this nose-type projection 49 moves into the slot 56. A second tensioning screw 57 is then screwable into the further tensioning means 51, for which purpose the further tensioning means 51 is provided with a corresponding inner thread 58 in the region of the through hole 54.

Shown in FIG. 5 is the further tensioning means 51 in the state in which the first connecting element 8 and the nose-type projection 49 are fixed in this further tensioning means 51. It can be seen here that the lower surface of the clamping element 55 is pressed on the first connecting element 8, which supports itself on the bottom of the U-shaped receiving part 52. The nose-type projection 49 projects into the slot 56 (FIG. 4) of the clamping element 55, which nose-type projection is held in a clamped way between the two crosspieces 59 and 60 of the clamping element 55, which crosspieces can move elastically toward one another. Via the second tensioning screw 57, which is screwed into and tightened in the inner thread 58 of the further tensioning means 51, the necessary gripping forces are applied for holding the first connecting element 8 and the nose-type projection 49 in the further tensioning means 51.

The great advantage of the vertebral column implant shown here consists in that after the growing together of the vertebral bodies fixed by means of the rigid first connecting element 8, this rigid reinforcement can be completely decoupled from the region which is stabilizing for the vertebral bodies by means of the flexible second connecting element 5. This flexible stabilized region should however continue to fulfill the stabilizing function. To decouple the rigid part from the elastic part of the vertebral column implant, the second tensioning screw 57 of the further tensioning means 51 can be loosened, the further tensioning means 51 can be pushed together with the clamping element 55 along the first connecting element 8 away from the second bone screw 2, <and> the clamping element 55 can be removed from the further tensioning means 51. Afterwards the tensioning means 23 of the second bone screw 2 receiving the end region 11 can be released. The tensioning element 24 can then be lifted out of this second bone screw 2 and axially thereto. Through the formfitting connection of the second connecting element 5 to the second bone screw 2 this connection remains stable; a tensioning means 23, whose tensioning element 24 is of “normal” design without nose-type projection 49, can then be placed on the bone screw 2. Thus in this way the rigid region of the vertebral column implant 1, after it has fulfilled its function, can be completely decoupled from the elastic region. A gap results between the first connecting element 8 and the second connecting element 5, so no forces can be transmitted anymore from one connecting element to the other connecting element. It is also conceivable that, after the growing together of the vertebral bodies fixed by means of the rigid first connecting element 8, the rigid first connecting element 8 and if necessary the first bone screws are removed from the body of the patient, which can take place accordingly by releasing the coupling means 9 and without adverse effects on the stabilizing, elastic region of the vertebral column implant; the elastic region of the vertebral column implant remains unchanged in the body.

The second embodiment of the vertebral column implant 1 according to the invention, as it is shown in FIGS. 8-11, differs from the initially described first embodiment example of a vertebral column implant only in that the coupling means 9 are designed differently. As can be learned from FIGS. 8, 10 and 11, the nose-type projection 49, which is provided on the tensioning element 24, consists of two crosspieces 59 and 60 running parallel. The clamping element 55 is composed of a base plate 61 and a cylindrical part 62. This clamping element 55 is once again insertable into the further tensioning means 51. The two crosspieces 59 and 60 end up again in the further tensioning means 51. With the second tensioning screw 57 the first connecting element 8 can be connected to the tensioning element 24. FIG. 9 shows that, in the assembled state, the first connecting element 8 is placed in the U-shaped receiving part and is supported thereon. The base plate 61 of the clamping elements 55 is pressed against the first connecting element 8, in that the two crosspieces 59 and 60 of the clamping element 55 are pressed against this base plate 61 by means of the second tensioning screw 57. An optimal fixing of the first connecting element 8 by means of the tensioning element 24 is thereby achieved here too. The decoupling of the rigid region of the vertebral column implant from the elastic region of the vertebral column implant in a simple way, as has been previously described, is possible here too.

The third embodiment example of a vertebral column implant 1 according to the invention is shown in FIGS. 12-15. This embodiment example differs once again through a differently designed coupling means 9 from the two previously described embodiment examples. As can be seen from FIGS. 12, 14 and 15, the tensioning element 24, which is insertable in the second bone screw 2, is provided once again with a nose-type projection 49. Compared with the nose-type projection 49 according to the first embodiment example, this is designed wider. A bore 63, which is provided with an inner threading, is made in this nose-type projection 49. Screwable into this threaded bore 63 is a further tensioning screw 64.

In the assembled state of this coupling means 9, as can be seen from FIG. 13, the first connecting element 8 is situated in the U-shaped receiving part 52 of the further tensioning means 51. The nose-type projection 49 projects into the U-shaped receiving part 52 of the further tensioning means 51. The further tensioning screw screwed into the threaded bore 63 of the nose-type projection 49 presses on the first connecting element 8. The second tensioning screw 57 presses, for its part, on the nose-type projection 49. The first connecting element 8 and the nose-type projection 49 are thereby fixed in a clamped way in the further tensioning means 51. The height position of the nose-type projection 49 with respect to the first connecting element 8 can be adjusted by corresponding screwing in of the further tensioning screw 64, whereby adaptations of the position of the first connecting element 8 with respect to the adjacent second bone screw 2 and thus to the second connecting element 5 can be carried out in an optimal way.

Also with this design of the coupling means 9, the rigid region of the vertebral column implant can be decoupled in the simplest way from the elastic stabilization region of the vertebral column implant, as has been previously described, in that the second tensioning screw 57 and the further tensioning screw 64 are screwed out and removed.

With this vertebral column implant according to the invention, as has already been mentioned, the advantage is obtained that after the ossification of vertebral bodies, which is achieved through a rigid region of a vertebral column implant, this rigid region can be decoupled in a simple way from the elastic stabilization region of this vertebral column implant. This would also make possible, if necessary, a simple removal of the rigid region of the vertebral column implant from the body without the stabilizing elastic region of the vertebral column implant being adversely affected.

1. A vertebral column implant for stabilization and stiffening of vertebral bodies of a vertebral column, comprising:
first bone screws, consisting of a screw-in portion, which is screwable into a vertebral body, and first receiving means;
second bone screws, consisting of a screw-in portion, which is screwable into a vertebral body, and second receiving means;
first connecting elements, which are rigid and are insertable in the first receiving means of the first bone screws and are able to be fixed therein;
second connecting elements, which are elastic and are insertable in the second receiving means of the second bone screws and are able to be fixed therein;
and coupling means, by means of which a first connecting element and a second connecting element are connectible to one another in each case,
wherein
the coupling means, with a first region, are able to be attached to a second bone screw in a releasable way, in which second bone screw an end region of a second connecting element is held, and
the coupling means with a second region opposite the first region are connectible to an end region of a first connecting element.

2. The vertebral column implant according to claim 1, wherein the first region of the coupling means is provided with a tensioning element, which is insertable in a guided way into the second receiving means and via tensioning means, which are able to be put on the second receiving means, is able to be tensioned against the second connecting element inserted in the second receiving means.

3. The vertebral column implant according to claim 2, wherein the second receiving means are designed U-shaped, and are composed of two arms, between which the bearing surface for the second connecting element is disposed and on which two arms the tensioning means, consisting of a locking element provided with a first tensioning screw, is able to be placed and locked therewith.

4. The vertebral column implant according to claim 3, wherein the tensioning element is provided with guide ribs, which, together with guide surfaces provided on the arms, form the guides.

5. The vertebral column implant according to claim 1, wherein the second region of the coupling means is designed as a further tensioning means, in which an end region of a first connecting element is able to be fixed.

6. The vertebral column implant according to claim 5, wherein the further tensioning means has a U-shaped receiving part for the first connecting element and a threaded part, opposite the U-shaped receiving part, for receiving a second tensioning screw.

7. The vertebral column implant according to claim 6, wherein inserted between the second tensioning screw and the first connecting element inserted in the further tensioning means is a clamping element.

8. The vertebral column implant according to claim 1, wherein the tensioning element and the further tensioning means, which form the coupling means, are connected to one another in a releasable way.

9. The vertebral column implant according to claim 8, wherein the tensioning element has at least one nose-type projection, which projects into the further tensioning means.

10. The vertebral column implant according to claim 9, wherein the nose-type projection is provided with a threaded bore, into which a further tensioning screw is screwable, which nose-type projection projects between the second tensioning screw and the first connecting element of the further tensioning means.

11. The vertebral column implant according to claim 1, wherein the second connecting elements have a surface structure consisting of ridges and grooves running substantially transversely to the longitudinal axis.

12. The vertebral column implant according to claim 11, wherein the second receiving means and the surface of the tensioning means turned toward the second connecting element are provided with grooves and ridges that correspond to the ridges and grooves of the second connecting element.
